(19) United States
(12) Patent Application Publication
(10) Pub. No.: US 2007/0184549 A1
(43) Pub. Date: Aug. 9, 2007

(54) Method of inducing fenestrae

(76) Inventors: David T. Shima, Barnet (GB); Sofia Ioannidou, Boston, MA (US)

Correspondence Address: (OSI) EYETECH, INC., 41 PINELAWN ROAD, MELVILLE, NY, 11747, US

(21) Appl. No.: 11/272,305

(22) Filed: Nov. 10, 2005

Related U.S. Application Data
(60) Provisional application No. 60/627,981, filed on Nov. 15, 2004.

(51) Int. Cl. C12N 5/06; A61K 31/407

(52) U.S. Cl. 435/325; 514/411

(57) ABSTRACT

The present invention relates to a method of inducing fenestrae in an endothelial cell line. More particularly, the present invention relates to inducing fenestrae in a bEND5 cell line or a Py4.1 cell line utilizing latrunculin A or cytochalasin B as an inducing agent.

### RELATED APPLICATION

This Application claims the benefit of U.S. Provisional Application No. 60/627,981, filed on Nov. 15, 2004. The entire teachings of the above application is incorporated herein by reference.

### FIELD OF THE INVENTION

The present invention relates to a method of inducing fenestrae in an endothelial cell line. More particularly, the present invention relates to inducing fenestrae in a bEND5 cell line or a Py4.1 cell line utilizing latrunculin A or cytochalasin B as an inducing agent.

### BACKGROUND OF THE INVENTION

Endothelial fenestrae were first described in the 1950s, but their composition, function, and biogenesis remain vastly unknown. Tissue complexity coupled to the lack of a rapid screening assay for the presence of fenestrae, renders their study in vivo inherently difficult. In vitro studies which normally provide ease of manipulation and characterization are not a preferred option, as cells that have fenestrae in vivo, become dedifferentiated and lose the fenestrated phenotype when placed in culture [1, 2]. As a result, the study of fenestrae has been limited to descriptive and morphological analyses at the ultrastructural level.

Recent years have seen improvements in the numbers of fenestrae observed in culture, through attempts to render culture conditions more physiological. The first demonstration of occasional fenestrae in vitro came from cloned endothelial cells that mimicked the three-dimensional appearance of capillaries in vivo, when grown in tumor-conditioned medium [3]. Subsequently, low levels of fenestrae induction have been achieved by culturing endothelial cells on epithelial cell—secreted extracellular matrix [1, 2, 4] or treating them with Vascular Endothelial Growth factor (VEGF) [1], a potent permeability mediator implicated in fenestrae formation in vivo [5, 6]. Fenestrae formation has also been stimulated by factors of no apparent physiological relevance such as phorbol myristate acetate (PMA) [7, 8], retinoic acid [9], and the actin disruption agents cytochalasin B and latrunculin A [10-13]. The highest numbers of fenestrae seen so far where in already fenestrated primary liver sinusoid endothelial cells induced 2-3 fold [10-12]. The maximum number of fenestrae attained in cloned endothelial cells was at the order of 0.05-0.2 fenestrae per μm²[1, 4, 7-9], which is much lower than what has been documented for fenestrated capillary beds in vivo.

### SUMMARY OF THE INVENTION

The present invention provides a method of inducing the formation of fenestrae in an endothelial cell line selected from the group consisting of a bEND5 endothelial cell line and a Py4.1 endothelial cell line comprising the step of administering an inducing agent selected from the group consisting of latrunculin A and cytochalasin B.

Applicants have identified an endothelial cell line that is susceptible to fenestrae induction in significant amounts. Applicants established optimal conditions for induction enabling the performance of cell biological and biochemical studies. The present invention provides the characterization of an in vitro culture system using quantitative ultrastructural methods, with a focus on the roles of cytoskeletal remodeling and cellular predisposition within fenestrae biogenesis.

The present invention presents the establishment of the first in vitro culture model for de novo fenestrae induction in quantities sufficient for cell biological and biochemical studies. The present invention provides a foundation for further cell biological and biochemical studies of fenestrae..

### BRIEF DESCRIPTION OF THE DRAWINGS

FIG. 1 shows images of bEND5 cells untreated (A) and induced with 2.5 μM Latrunculin A (B) examined using an SEM. A large number of vesicles are present on the continuous plasma membrane of untreated cells (A). In induced cells, the plasma membrane is perforated by numerous fenestrae, that appear as regularly arranged discontinuities with a diameter of 70 nm (B). (Bar=1 μm)

FIG. 2 shows images of bEND5 cells untreated (A) and induced with 2.5 μM latrunculin A (B) examined using the wholemount TEM method. A continuous plasma membrane in untreated cells (A) is replaced by numerous well-ordered fenestrae upon induction (B). A trace of the diaphragm spanning each fenestra can be seen. (Bar=500 nm)

FIG. 3 shows stereology applied to SEM images. Images were printed, and sieve plates were marked (solid line). A grid (20 mm×20 mm) was overlaid over each image and the number of grid points falling within the areas delineated or on any cellular structure were counted. Fenestrae abundance was estimated based on the following calculation: Plasma membrane coverage (or fraction of the plasma membrane occupied by sieve plates)=grid points falling on sieve plates/grid points falling on any cell area. To express abundance in terms of the number of fenestrae per area of plasma membrane, the density of fenestrae within sieve plates was calculated and multiplied by the frequency of sieve plates as follows: number of fenestrae per square μm=density of fenestrae×plasma membrane coverage.

FIG. 4 is a graph regarding quantitation of fenestrae induction. bEND5 cells were grown on gelatin and treated with VEGF (75 ng/ml) for 6, 16, 25, 40, or 90 hours, with the addition of cytochalasin B (10 μM) for the last 2 hours of each time point. The number of fenestrae was assessed by SEM. Data points represent the average of 30 images captured per time point.

FIG. 5 is a graph regarding kinetics of fenestrae induction by microfilament disruption. bEND5 cells were grown on gelatin, treated with Cytochalasin B (10 μM) or Latrunculin A (2.5 μM) for a variety of time points, and the number of fenestrae quantified by SEM. Data points represent averages of two experiments, with 20 images taken per time point in each experiment. Error bars represent the standard deviation between the two experiments. For the 120 minute timepoint of the latrunculin A treatment, data was available from only one experiment.

FIG. 6 shows an image of a bEND5 cell. A quarter of the total surface area of a bEND5 cell induced with Latrunculin A as seen under low power SEM. The majority of the plasma membrane is fenestrated, apart from the nucleus and cytoplasmic arms between sieve plates. (Bar=1 μm)

FIG. 7 shows images of intermediates observed by SEM in a time-course of fenestrae induction using microfilament disruption agents: Small sieve plates (A), Depressions of large patches of the plasma membrane (B), Fenestrae with incomplete perforation (C), Diaphragmed vesicles clustered near flat fenestrated areas (D). (Bar=1 μm)

FIG. 8 shows images comparing fenestrae formation in different endothelial cell subtypes treated with 2.5 μM Latrunculin A. Abundant fenestrae are present in bEND5 (A) and Py4.1 (B) treated cells. No fenestrae are present in SVE cells (C), while rare fenestrae (arrowheads) are detected in HUVECs (D). (Bar=1 μm)

FIG. 9 is a graph regarding fenestrae induction in different endothelial cell types. The potential of different endothelial cell lines to form fenestrae under the optimal parameters of induction was assessed using wholemount TEM. bEND5, Py4.1, SVEC, and HUVEC cells were treated for 3 hours with vehicle (V) or 2.5 μM Latrunculin A (Lat), and the number of fenestrae was estimated as a fraction of the plasma membrane area occupied by sieve plates. Data points represent the average of two experiments, with 36 images captured per sample in each experiment. Error bars represent standard deviation between the two experiments.

FIG. 10 shows images of bEND5 cells treated with 10 μM Cytochalasin B for different periods of time, and actin microfilaments stained with Alexa546-conjugated phalloidin. Stress fibers were reduced within the first 20 minutes, whereas cortical actin persisted until about 60 minutes of treatment. At 120 minutes, individual actin bundles were spread around the cytoplasm. (Bar=8 μm)

FIG. 11 show images of bEND5 cells that were treated with 2.5 μM Latrunculin A for different periods of time, and actin microfilaments were stained with Alexa546-conjugated phalloidin. Stress fibers were reduced within the first 20 minutes, while complete disruption occurs at 60-120 minutes. Individual actin foci, small filaments, and actin-rich filopodia were present after 180 minutes of treatment. (Bar=8 μm)

FIG. 12 shows images of bEND5cells. bEND5 (A, B), Py4.1 (C, D), SVEC (E, F), and HUVEC (G, H) endothelial cells were treated with 2.5 μM Latrunculin A (B, D, F, H) or vehicle alone (A, C, E, G) for 3 hours, and their actin cytoskeleton was examined by staining with Alexa546—conjugated phalloidin. Microfilaments were disrupted in all cell types upon treatment. (Bar=8 μm)

FIG. 13 shows SEM Immunolabeling for PV-1(A) and PECAM (C), using 10 nm and 5 nm gold conjugated secondary antibodies respectively. PV-1 is seen to localize specifically on the diaphragm of fenestrae, while PE-CAM is distributed throughout the surface of the cell. Gold particles were visualized in SEM backscatter mode (A, C), while the corresponding secondary emission image (SET) is shown in B and D. (Bar=100 nm)

### DETAILED DESCRIPTION OF THE INVENTION

In one aspect, the invention provides a method of inducing the formation of fenestrae in an endothelial cell line selected from the group consisting of a bEND5 endothelial cell line and a Py4.1 endothelial cell line comprising the step of administering an inducing agent selected from the group consisting of latrunculin A and cytochalasin B.

In one embodiment, the inducing agent induces the formation of fenestrae in an amount greater than or equal to about 10-fold from an un-induced endothelial cell line. In another embodiment, the inducing agent induces the formation of fenestrae in an amount greater than or equal to about 20-fold from an un-induced endothelial cell line. In another embodiment, the inducing agent induces the formation of fenestrae in an amount greater than or equal to about 50-fold from an un-induced endothelial cell line. In another embodiment, the inducing agent induces the formation of fenestrae in an amount greater than or equal to about 100-fold from an un-induced endothelial cell line. In another embodiment, the inducing agent induces the formation of fenestrae in an amount greater than or equal to about 200-fold from an un-induced endothelial cell line.

In one embodiment, the inducing agent induces the formation of fenestrae to greater than or equal to about 100 fenestrae per cell. In another embodiment, the inducing agent induces the formation of fenestrae to greater than or equal to about 1000 fenestrae per cell.

In one embodiment, the inducing agent induces the formation of fenestrae to greater than or equal to about 0.1 fenestrae per μm². In another embodiment, the inducing agent induces the formation of fenestrae to greater than or equal to about 1.0 fenestrae per μm². In another embodiment, the inducing agent induces the formation of fenestrae to greater than or equal to about 3 fenestrae per μm². In another embodiment, the inducing agent induces the formation of fenestrae to greater than or equal to about 5 fenestrae per μm². In another embodiment, the inducing agent induces the formation of fenestrae to greater than or equal to about 60 fenestrae per μm².

In one particular embodiment, the inducing agent induces the formation of fenestrae to about 3.5 fenestrae per μm². In another particular embodiment, the inducing agent induces the formation of fenestrae to about 4.5 fenestrae per μm². In another particular embodiment, the inducing agent induces the formation of fenestrae to about 5.3 fenestrae per μm².

In one embodiment, the endothelial cell line is a bEND5 endothelial cell line. In another embodiment, the endothelial cell line is a Py4.1 endothelial cell line.

In one embodiment, the inducing agent is latrunculin A. In another embodiment, the inducing agent is cytochalasin B.

In one embodiment the invention provides a method of inducing the formation of fenestrae comprising the steps of administering latrunculin A to a bEND5 cell line.

In another embodiment the invention provides a method of inducing the formation of fenestrae comprising the step of administering latrunculin A to a Py4.1 cell line.

In another embodiment the invention provides a method of inducing the formation of fenestrae comprising the steps of administering cytochalasin B to a bEND5 cell line.

In another embodiment the invention provides a method of inducing the formation of fenestrae comprising the step of administering cytochalasin B to a Py4.1 cell line.

In another embodiment the invention provides a method of inducing the formation of fenestrae in a bEND5 cell line comprising the steps of:

a) administering cytochalasin B to the bEND5 cell line; and

b) administering VEGF-A to the bEND5 cell line.

In another embodiment the invention provides a method of inducing the formation of fenestrae in a bEND5 cell line comprising the steps of:

a) administering cytochalasin B to the bEND5 cell line; and

b) administering PMA to the bEND5 cell line.

In vitro Model for Fenestrae Formation

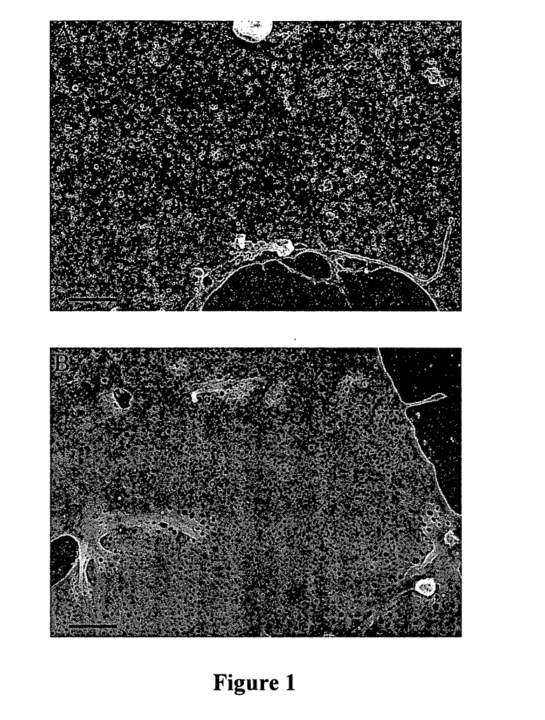

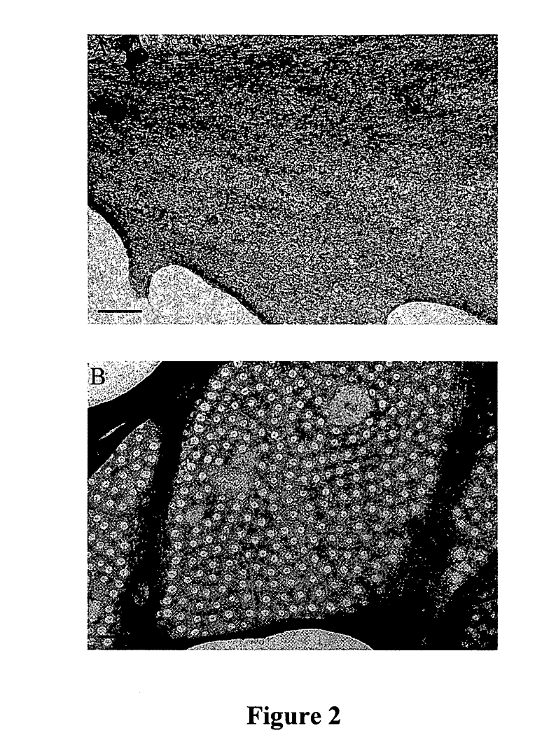

Following systematic screening through a panel of cloned endothelial cell lines cultured on a gelatin extracellular matrix and induced with a combination of PMA/cytochalasin B, or VEGF-A/cytochalasin B, the bEND5 brain endothelioma cell line was identified as capable of forming fenestrae. The presence of fenestrae was assessed using Scanning Electron Microscopy (SEM) (FIG. 1), or Wholemount Transmission Electron Microscopy (TEM) (FIG. 2). Under basal conditions, no or very few fenestrae were observed on the plasma membrane of bEND5 cells (FIGS. 1A, 2A). After induction, a significant portion of the plasma membrane was perforated by numerous fenestrae that occurred in sieve plates with a high degree of order: they displayed consistent diameters, linear arrangement and equidistant spacing (FIGS. 1B, 2B).

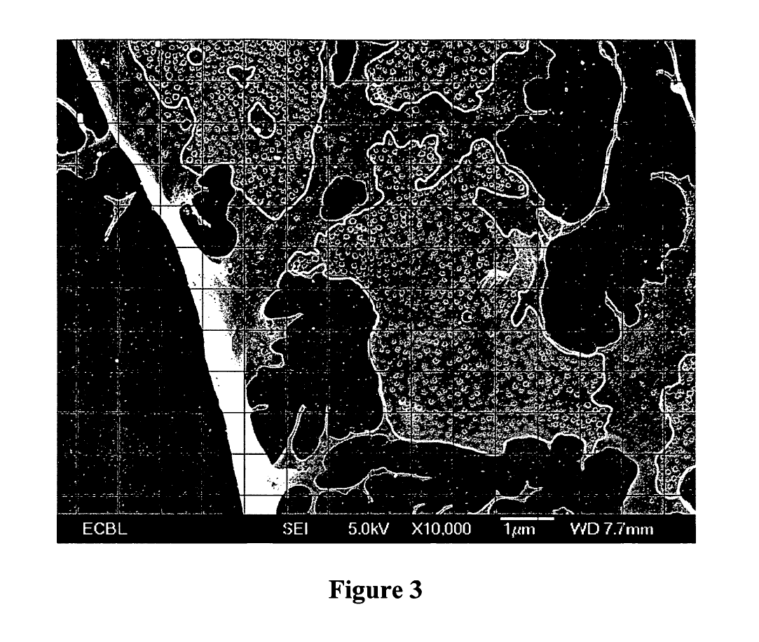

A quantitative assessment of fenestrae numbers was achieved by applying Stereology to SEM or wholemount TEM images (FIG. 3). Quantitative assessment comprised overlaying a lattice on top of an image and counting points falling on fenestrated and on non-fenestrated areas of the plasma membrane. Amounts of fenestrae were routinely expressed as fractions of plasma membrane area that was fenestrated (point counts falling on fenestrae divided by point counts falling on any plasma membrane area). To compare these numbers with measurements in previous in vivo or in vitro studies, the density of individual fenestrae within sieve plates was estimated, which when multiplied by the fraction of plasma membrane coverage equaled the number of fenestrae per μm² of plasma membrane. In order to minimize ambiguities in the quantitation, caused by similarities between various membrane structures as seen by EM, the following criteria where set: 1) Fenestrae were only considered if they occurred in sieve plates; and 2) Sieve plates were defined as groups of more than 10 fenestrae, having some degree of order, and residing in attenuated regions near the edges of cells.

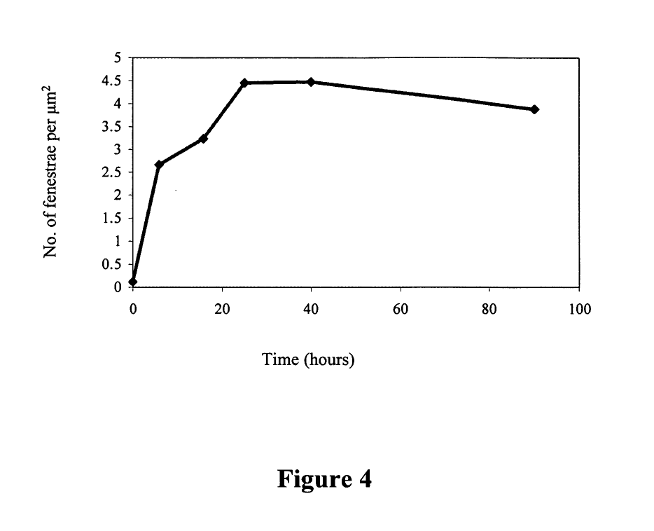

To validate the quantitative potential of the model, a time-course of induction was performed using VEGF-A (75 ng/μl) over a period of 90 hours, with the addition of cytochalasin B (10 μM) for the last 2 hours of each time-point assessed (FIG. 4). The maximum number of fenestrae was reached after 24 hours in culture, and represented a 20-fold induction compared to the untreated control, with 4.5 fenestrae per μm², analogous to 100-1000s of fenestrae per cell. Omitting Cytochalasin B from the induction cocktail in some time points illustrates its role as the main determinant of fenestrae induction: Cells treated only with VEGF show minimal induction over background.

Identifying the optimal parameters for induction

The effect of cytoskeletal disruption

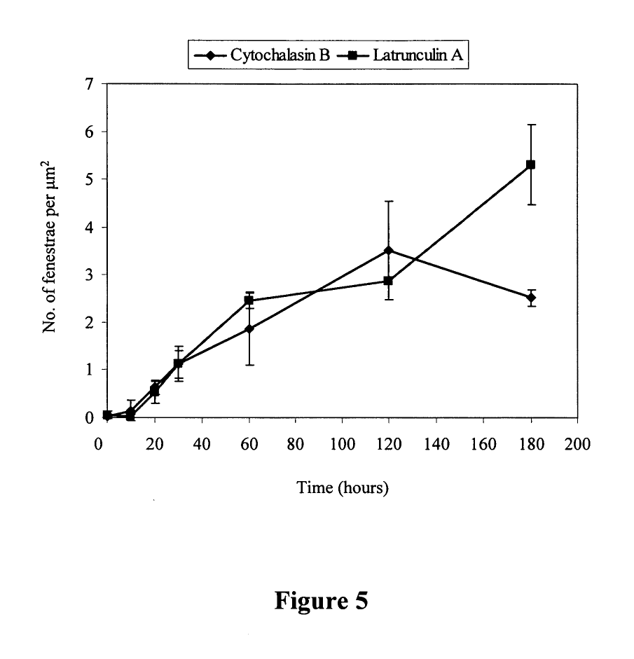

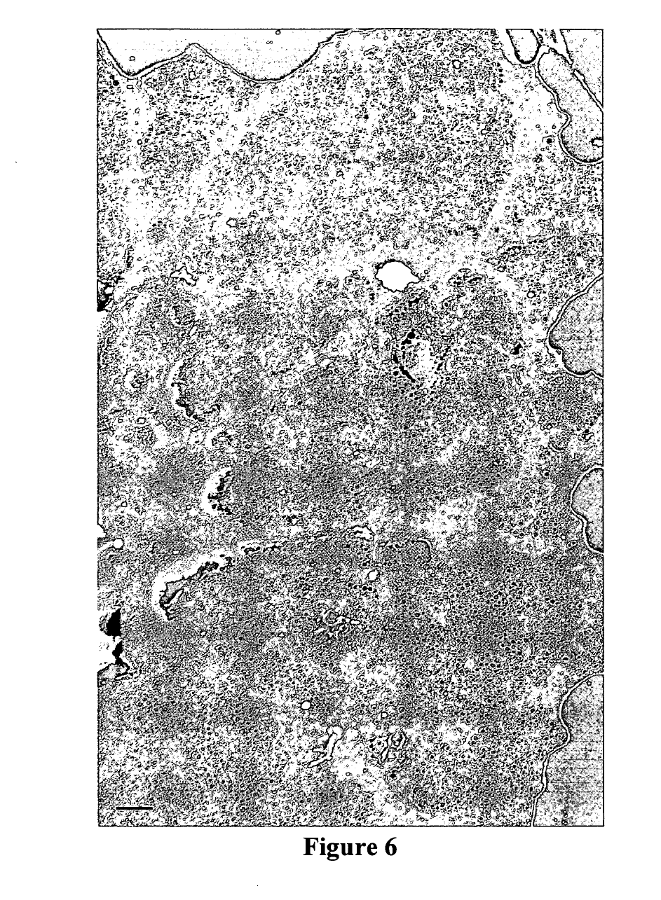

Two separate actin disruption agents, cytochalasin B (10 nM) and Latrunculin A (2.5 nM), were assessed in terms of their fenestrae-promoting potentials by SEM. Experiments focused on the early events of a short induction protocol to isolate primary from secondary events in fenestrae formation (FIG. 5). Both agents were able to stimulate induction in the order of 100-fold compared to the untreated control; their kinetics of induction, however, were different. Fenestrae started forming as early as after 20 or 30 minutes of treatment with Cytochalasin B. The maximum number of fenestrae (3.5 per μm²) was reached at 2 hours while there was a slight decrease in the number of fenestrae at 3 hours of treatment. Latrunculin A led to a maximum of 5.3 fenestrae per μm² after 3 hours of treatment. The initial kinetics of induction resembled those of cytochalasin B, but comparatively more fenestrae started to form with incubations over 30 minutes. The same amount of fenestrae was observed when treatments were extended over 3 hours, though the viability of the cells was reduced considerably. More cells responded to latrunculin A than to cytochalasin B. Within those cells, the majority of their surface area was fenestrated, with the exception of the nucleus and some thick areas separating sieve plates that could be accommodating microtubules [12] or actin filament remnants (FIG. 6). From a lower magnification, cells appeared like ‘fried eggs’ decorated with a network of filopodia.

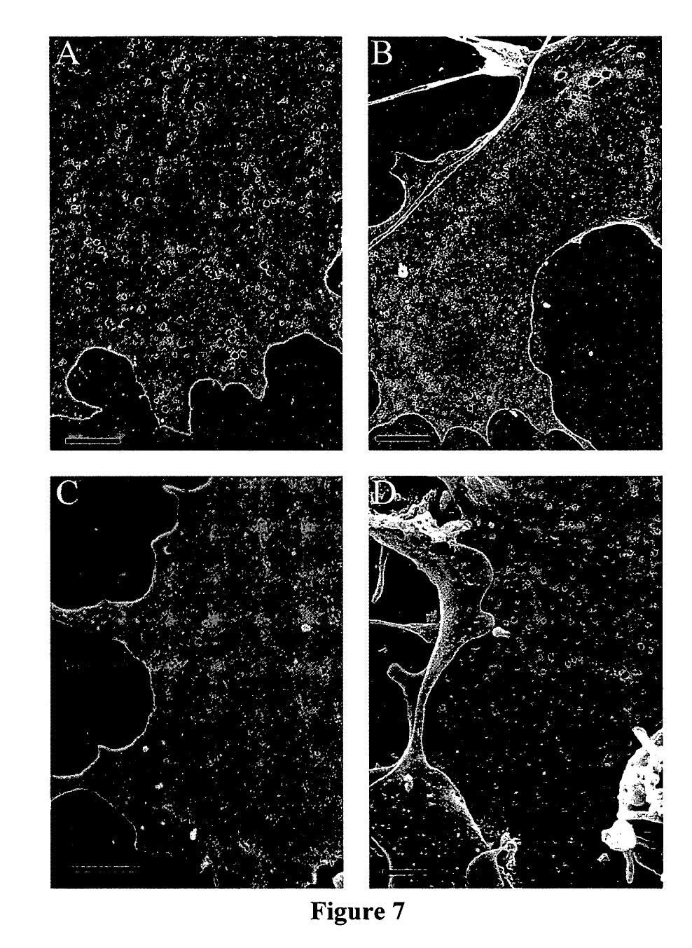

In an attempt to understand fenestrae biogenesis, early points in the induction time-course were examined. Overall, cells changed from having no fenestrae to having few isolated sieve plates (FIG. 7A), and then to having large sieve plates covering the majority of their surface. A number of intermediates were consistently spotted, sometimes overlapping in distant time points. Often depressions of plasma membrane were observed (FIG. 7B). Sieve plates with fenestrae-like structures that did not span the cytoplasm of the cell were identified (FIG. 7C). Fenestrae were often observed near caveolae or other unidentified endocytic structures (FIG. 7D).

The Effect of Endothelial Cell Subtypes

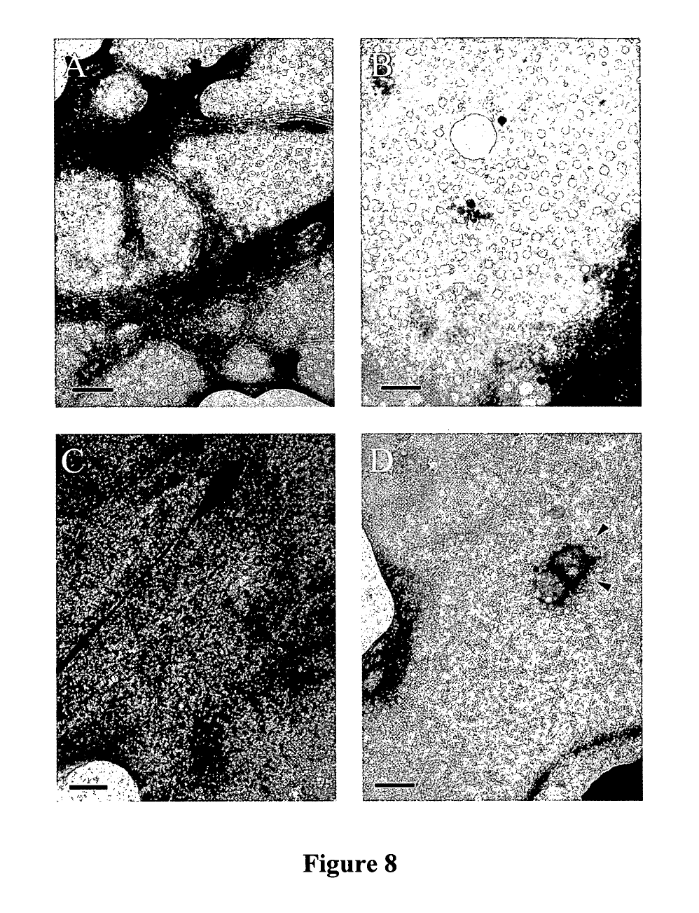

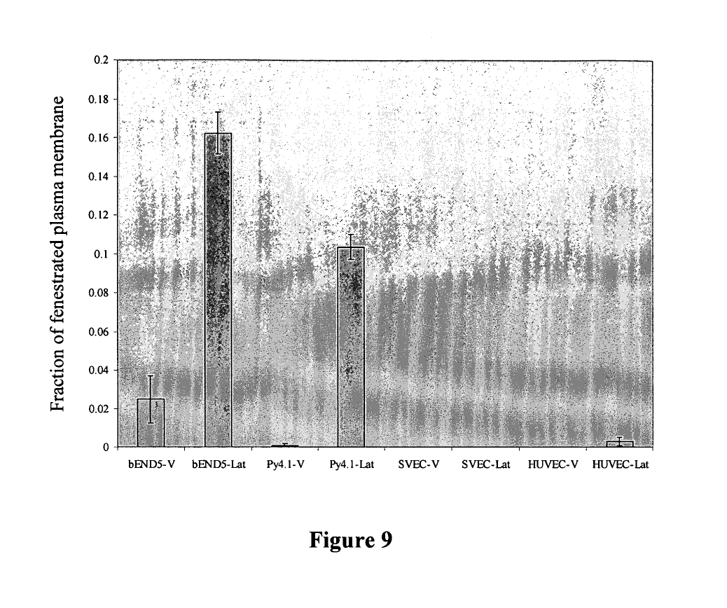

A panel of endothelial cell subtypes was assessed for their fenestrae-forming potentials. Three mouse endothelial lines (bEND5, Py4.1, SVEC) and one human primary endothelial line (HUVEC) were cultured on gelatin-coated grids, treated with latrunculin A for 3 hours, and examined by wholemount TEM (FIG. 8). bEND5 and Py4.1 cells are brain endothelomas and ear/tail endotheliomas respectively, both originating from hemangiomas of Polyoma virus—infected mice. SVECs are lymph node endothelial cells infected with SV40 virus. HUVECs were chosen because of their widespread use and primary cell nature. Quantitation of the response for each cell line, showed that only Polyoma middle T-containing bEND5 and Py4.1 cells were susceptible to fenestrae formation, with SVECs showing no indication of fenestrae, and HUVECs producing only very few (FIG. 9). The response of bEND5 and Py4.1 cells differed both in magnitude and in relation to the respective untreated controls. bEND5 cells harbored greater numbers of fenestrae overall, but contained some background fenestrae in the un-induced state. Py4.1 cells harbored lower absolute numbers of fenestrae, but showed a greater relative induction than bEND5 cells, due to the absence of fenestrae in the uninduced state. Py4.1 cells were found to be responsive to cytochalasin B.

Actin microfilaments under the light microscope

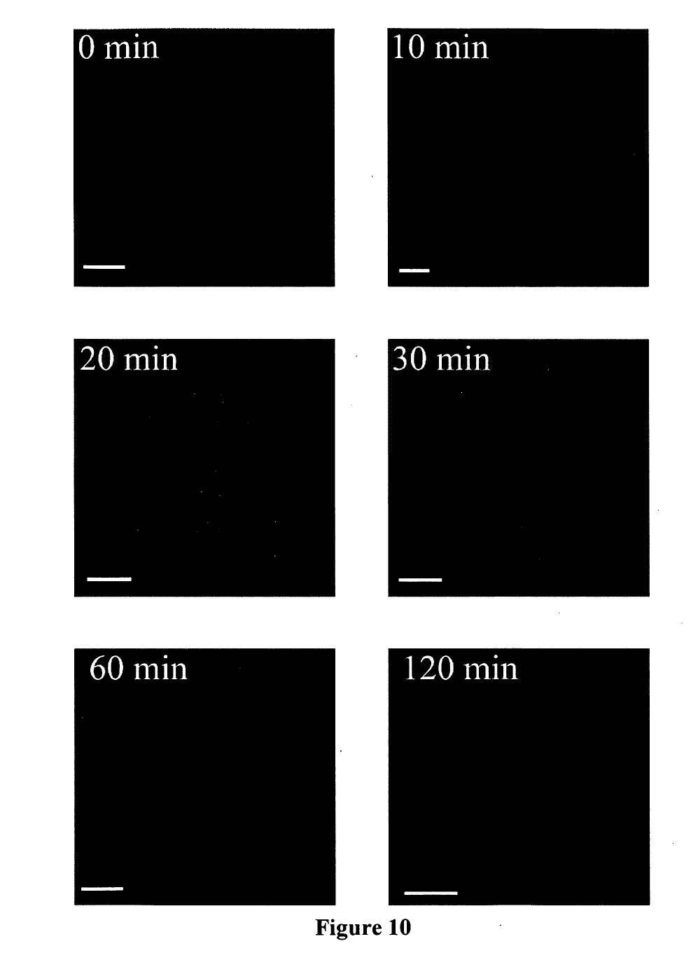

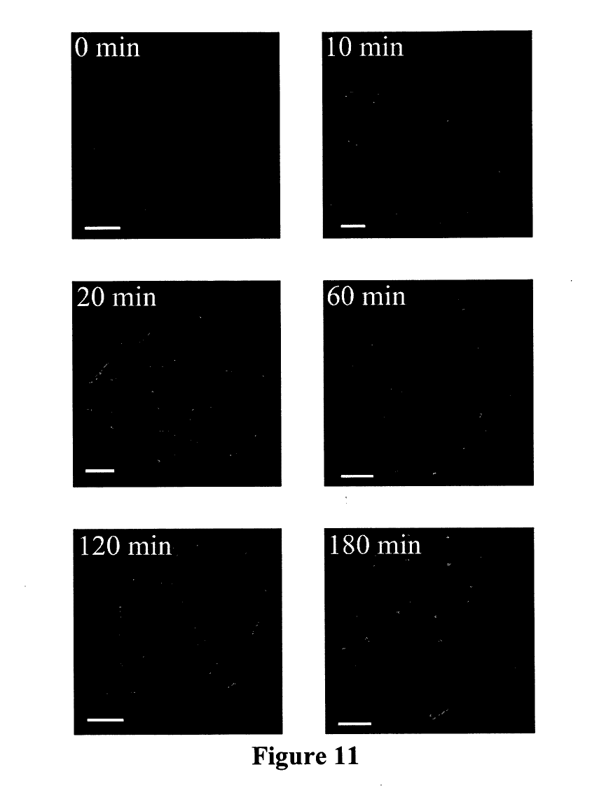

In parallel to the SEM analysis, bEND5 cells subjected to cytochalasin B and latrunculin A time-courses were stained for the presence of actin microfilaments using phalloidin (FIGS. 10 and 11). Untreated cells contained an extensive network of stress fibers spanning their cytoplasm. Dramatic changes in the organization of actin were however observed as early as after 10 minutes of treatment with either agent. Cells treated with cytochalasin B (FIG. 10) exhibited a gradual decrease in the amount of stress fibers between the time points of 10, 20, and 30 minutes, with cortical actin remaining in place until about 60 minutes of treatment. At 120 minutes, the disassembled actin was concentrated in aggregates throughout the cytoplasm, while large areas remained actin-deficient. In cells treated with latrunculin A (FIG. 11) the disruption of stress fibers was faster, and the cortical cytoskeleton was dismantled within the first 20 minutes. Overall cells had more and larger actin-depleted areas, and the remainder of actin appeared irregularly arranged in short filaments around the cytoplasm or in structures resembling filopodia. The appearance of actin-depleted areas under light microscopy, correlated well with the emergence of fenestrae under scanning electron microscopy, and suggested a structural or a functional relationship between the two processes.

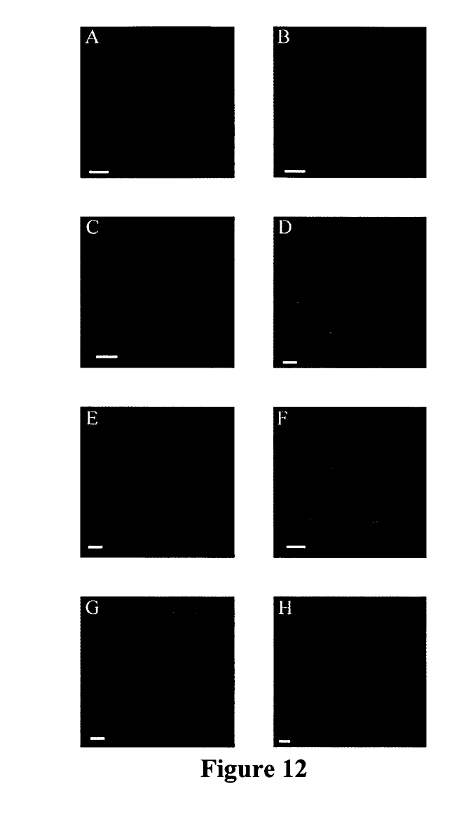

The actin cytoskeleton of all four cell types was stained with phalloidin to study the differential induction of endothelial cell subtypes by latrunculin A (FIG. 12). In all cases, the cytoskeleton was completely disrupted, confirming the non-discriminatory effect of the drug, and suggesting the requirement for additional factors in order for fenestrae formation to take place. Observation of bEND5 and Py4.1 cells highlighted a difference in their actin cytoskeleton in the steady state (FIGS. 12A and 12C). Py4.1 cells had greater amounts of cortical actin. This could account for their decreased susceptibility to fenestrae formation compared to bEND5 cells, a discrepancy particularly apparent during induction with cytochalasin B.

Immunogold labeling at the SEM level

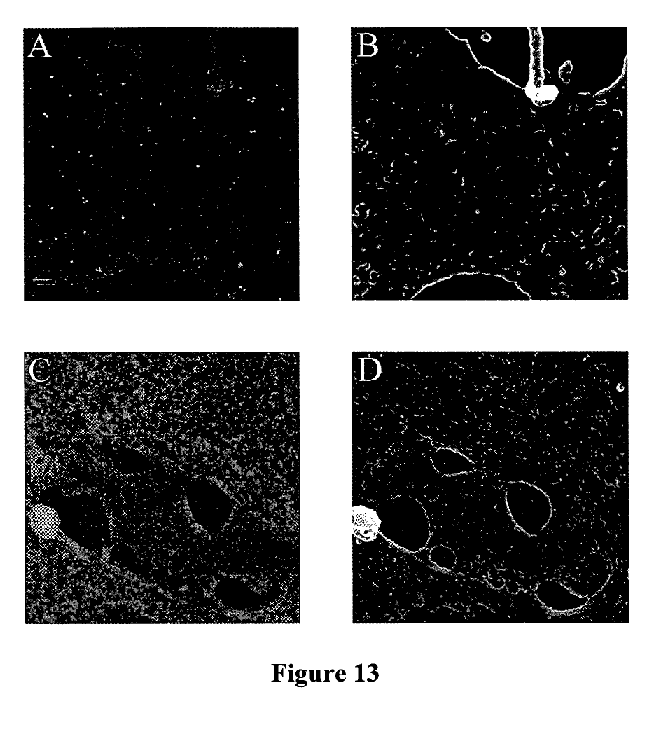

Fenestrae induced in bEND5 or Py4.1 cells appeared morphologically similar to fenestrae described in vivo. Observed under high power, they had a consistent circular diameter of about 70 nm and were usually spanned by a diaphragm. Immunogold labeling was performed using an antibody against the protein PV-1, a shared component of the diaphragms of fenestrae and caveolae, and the only known constituent of fenestrae to date [14, 15]. PV-1 labeling was found to concentrate on sieve plates of induced bEND5 cells, and specifically on the diaphragms of fenestrae (FIG. 13A-B). In contrast, immunogold labeling with an anti-PECAM antibody showed no preference for any particular region of the cell, and was distributed in fenestrated and non-fenestrated areas alike (FIG. 13C-D).

A versatile model to study fenestrae biogenesis

One advantage of the present invention, a successful in vitro culture model for fenestrae formation is established by means of pairing endothelial cell types and induction stimuli. In one particular embodiment, bEND5 cells treated with latrunculin A, fenestrae were induced 100-fold at levels of up to 5.3 fenestrae per μm². This compared favorably to previous reports of in vitro studies where adrenal cortex endothelial cells (ACEs) or HUVECs, induced with VEGF, phorbol esters, or retinoic acid, attained maximal levels of only 0.187 fenestrae per μm² [1, 4, 7-9]. Studies with primary liver endothelial cells and the cytoskeleton disruption agent swinholide A reported fenestrae levels of up to 9.1 per μm², however, this represented a less than 3-fold induction, as the untreated control contained over 3 fenestrae per μm²[13]. Moreover, this enhancement of an already existing phenotype was only possible in freshly-isolated endothelial cells from the liver sinusoids as attempts to immortalize them rendered them no longer susceptible to fenestrae formation [16]. Comparisons to the numbers of fenestrae observed in vivo are more complicated, as reported levels of fenestrae vary between as low as 0.58 per μm²[17] and as high as 60 per μm² (Frederici 1968), depending on the capillary bed investigated and even more on the extent and type of biological sampling conducted.

Actin remodeling as a driving force for fenestrae formation

The central role for actin remodeling in sustaining or increasing the number of fenestrae had been highlighted in studies involving in vitro cell culture [10-13] and ex vivo organ culture [18], and was exploited in our system for the formation of de novo fenestrae. Cytochalasin B and Latrunculin A, both target the actin cytoskeleton, but achieve disruption through different mechanisms. Cytochalasin B belongs to a family of mold metabolites that inhibit the elongation of actin filaments by binding to their barbed, fast growing end with high affinity (Kd˜10⁻⁷-10⁻⁸ M)[19, 20]. It is thought to prevent monomer addition, without however decreasing the concentration of polymerized actin [21]. Latrunculin A belongs to a family of marine sponge toxins, which act by forming 1:1 complexes with actin monomers (Kd˜10⁻⁷ M), and thereby decreasing the concentration of actin filaments [22-24]. In agreement with the studies on liver sinusoidal endothelial cells [11], latrunculin A was more potent in inducing fenestrae than cytochalasin B, even when used at lower concentrations. The fact that two drugs with different mechanisms of action, but similar end-results on the state of actin, both led to the induction of fenestrae, in two independent endothelial culture models, supports the theory that fenestrae formation being linked to stress-fiber disassembly and not some side-effect of the drug. Moreover, in the culture model, the different extent to which microfilament disassembly was achieved with either agent, correlated with the magnitude of the response in terms of fenestrae formation. Treatment with latrunculin A resulted in greater disruption and also in more fenestrae.

The kinetics of fenestrae induction through cytoskeleton disruption were rapid, with fenestrae being induced in the first 20 minutes. While not wishing to be bound by theory, it is suggested that in this particular system, in a similar fashion to the already fenestrated liver endothelial cells, the components required for fenestrae formation were already present in the cell and were merely rearranged to form pores. The increase in fenestrae formation observed over time may be explained by a progression in actin disruption, either within single cells, or in the cell population. Further support for such a fast assembly of fenestrae components comes from in vivo studies showing that VEGF can induce fenestrations in certain normally non-fenestrated anatomical sites, within 10 minutes of topical application or intradermal injection [5].

### REFERENCES

1. Esser, S., et al., Vascular endothelial growth factor induces endothelial fenestrations in vitro. J Cell Biol, 1998. 140(4): p. 947-59.
2. Carley, W. W., A. J. Milici, and J. A. Madri, Extracellular matrix specificity for the differentiation of capillary endothelial cells. Exp Cell Res, 1988. 178(2): p. 426-34.
3. Folkman, J. and C. Haudenschild, Angiogenesis in vitro. Nature, 1980. 288(5791): p. 551-6.
4. Milici, A. J., M. B. Furie, and W. W. Carley, The formation of fenestrations and channels by capillary endothelium in vitro. Proc Natl Acad Sci U S A, 1985. 82(18): p. 6181-5.
5. Roberts, W. G. and G. E. Palade, Neovasculature induced by vascular endothelial growth factor is fenestrated. Cancer Res, 1997. 57(4): p. 765-72.
6. Breier, G., et al., Expression of vascular endothelial growth factor during embryonic angiogenesis and endothelial cell differentiation. Development, 1992. 114(2): p. 521-32.
7. Lombardi, T., et al., Endothelial diaphragmed fenestrae: in vitro modulation by phorbol myristate acetate. J Cell Biol, 1986. 102(5): p. 1965-70.
8. Lombardi, T., R. Montesano, and L. Orci, Phorbol ester induces diaphragmed fenestrae in large vessel endothelium in vitro. Eur J Cell Biol, 1987. 44(1): p. 86-9.
9. Lombardi, T., et al., In vitro modulation of endothelial fenestrae: opposing effects of retinoic acid and transforming growth factor beta. J Cell Sci, 1988. 91 ( Pt 2): p. 313-8.
10. Steffan, A. M., J. L. Gendrault, and A. Kim, Increase in the number of fenestrae in mouse endothelial liver cells by altering the cytoskeleton with cytochalasin B. Hepatology, 1987. 7(6): p. 1230-8.
11. Braet, F., et al., Microfilament-disrupting agent latrunculin A induces and increased number of fenestrae in rat liver sinusoidal endothelial cells: comparison with cytochalasin B. Hepatology, 1996. 24(3): p. 627-35.
12. Braet, F., et al., Comparative scanning, transmission and atomic force microscopy of the microtubular cytoskeleton in fenestrated liver endothelial cells. Scanning Microsc Suppl, 1996. 10: p. 225-35; discussion 235-6.
13. Braet, F., et al., A novel structure involved in the formation of liver endothelial cell fenestrae revealed by using the actin inhibitor misakinolide. Proc Natl Acad Sci U S A, 1998. 95(23): p. 13635-40.
14. Stan, R. V., et al., Isolation, cloning, and localization of rat PV-1, a novel endothelial caveolar protein. J Cell Biol, 1999. 145(6): p. 1189-98.
15. Stan, R. V., M. Kubitza, and G. E. Palade, PV-1 is a component of the fenestral and stomatal diaphragms in fenestrated endothelia. Proc Natl Acad Sci U S A, 1999. 96(23): p. 13203-7.
16. Steffan, A. M., et al., Mouse hepatitis virus type 3 infection provokes a decrease in the number of sinusoidal endothelial cell fenestrae both in vivo and in vitro. Hepatology, 1995. 22(2): p. 395-401.
17. Milici, A. J., N. L'Hemault, and G. E. Palade, Surface densities of diaphragmed fenestrae and transendothelial channels in different murine capillary beds. Circ Res, 1985. 56(5): p. 709-17.
18. Andrews, P. M., Investigations of cytoplasmic contractile and cytoskeletal elements in the kidney glomerulus. Kidney Int, 1981. 20(5): p. 549-62.
19. MacLean-Fletcher, S. and T. D. Pollard, Mechanism of action of cytochalasin B on actin. Cell, 1980. 20(2): p. 329-41.
20. Cooper, J. A., Effects of cytochalasin and phalloidin on actin. J Cell Biol, 1987. 105(4): p. 1473-8.
21. Yahara, I., et al., Correlation between effects of 24 different cytochalasins on cellular structures and cellular events and those on actin in vitro. J Cell Biol, 1982. 92(1): p. 69-78.
22. Coue, M., et al., Inhibition of actin polymerization by latrunculin A. FEBS Lett, 1987. 213(2): p. 316-8.
23. Morton, W. M., K. R. Ayscough, and P. J. McLaughlin, Latrunculin alters the actin-monomer subunit interface to prevent polymerization. Nat Cell Biol, 2000. 2(6): p. 376-8.
24. Spector, I., et al., Latrunculins: novel marine toxins that disrupt microfilament organization in cultured cells. Science, 1983. 219(4584): p. 493-5.

### EXAMPLES

The following examples serve to illustrate certain useful embodiments and aspects of the present invention and are not to be construed as limiting the scope thereof. Alternative materials and methods can be utilized to obtain similar results.

All chemicals were purchased from Sigma-Aldrich, and Fluka, unless otherwise indicated. Phosphate buffer Saline without calcium or magnesium (PBS), LB medium, LB-agar, EDTA, trypsin/versene, glutamine, penicillin/streptomycin, Leibovitz L-15 medium were provided by CRUK or Eyetech Research Center central services.

### Example 1

Mammalian Tissue Culture

Isolation of Mouse Embryonic Fibroblasts

14.5 day old embryos were dissected from the extraembryonic membranes in Dulbeccos Modified Eagle's Medium (DMEM; Invitrogen). The liver was discarded, the head was removed for genotyping and the remainder of the embryo was trypsinised by incubating with 1 ml of trypsin/versene for 45 minutes on a shaker set at 37° C. Using a plastic Pasteur pipette, the embryo was dissociated by pipetting up and down five times, and split between two 60 mm dishes.

Maintenance of Mammalian Cell Lines

| Cell Line | Species | Origin | Passage No | Culture conditions |
|---|---|---|---|---|
| bEND5 | mouse | brain | 13-25 | DMEM high glucose with sodium pyruvate, |
| | | endothelioma | | 10% FBS, 4 mM L-glutamate, penicilin/streptomycin, |
| | | | | 5 μM β-mercaptoethanol, non-essential amino acids. |
| | | | | 37° C. incubator with 10% CO2 |
| Py4.1 | mouse | ear and tail | | DMEM high glucose with sodium pyruvate, 2% FBS, |
| | | hemangiomas | | penicilin/streptomycin. 37° C. incubator with |
| | | | | 10% CO2 |
| NIH 3T3 | mouse | embryo | | DMEM high glucose with sodium pyruvate, 10% |
| (ATCC) | | | | FBS, 4 mM L-glutamate, penicilin/streptomycin, |
| | | | | 1.5 g/L sodium bicarbonate. 37° C. incubator with |
| | | | | 5% CO2 |
| HUVEC | human | umbilical | 3-5 | M200, low supplement growth serum, |
| (Cascade | | vein | | penicilin/streptomycin (Cascade Biologics). 37° C. |
| Biologics) | | | | incubator with 5% CO2 |
| SVEC4-10 | mouse | lymph node | 3-5 | DMEM high glucose with sodium pyruvate, 10% |
| (ATCC) | | | | FBS, 4 mM L-glutamate, penicilin/streptomycin, |
| | | | | 1.5 g/L sodium bicarbonate. 37° C. incubator with |
| | | | | 10% CO2 |
| MEF | mouse | embryo | 2-3 | DMEM high glucose with sodium pyruvate, 10% |
| (mouse | | | | FBS Tet system approved (Clontech), 4 mM L- |
| embryonic | | | | glutamate, 2 × penicilin/streptomycin, 1.25 μg/ml |
| fibroblasts) | | | | Fungizone. 37° C. incubator with 10% CO2 |
| MEF 3T3 Tet- | mouse | embryo | 3 | DMEM high glucose with sodium pyruvate, 10% |
| Off cell line | | | | FBS Tet system approved (Clontech), 4 mM L- |
| (Clontech) | | | | glutamate, penicilin/streptomycin. 37° C. incubator |
| | | | | with 10% CO2 |

All culture media and related products were obtained from Invitrogen, unless otherwise indicated.

All cell lines were trypsinised using trypsin/versene solution. Alternatively cell lines were trypsinized using 1× Trypsin EDTA solution (Invitrogen), apart from bEND5 and Py4.1 cells which required 10× Trypsin EDTA solution (Invitrogen), diluted 1:1 with DMEM.

Cells were thawed by diluting the contents of an ampule in 9 volumes of medium, sedimenting the cells at 300 g and resuspending the pellet in the appropriate volume of complete medium. Cells were frozen in 10% DMSO, 20% Fetal Bovine Serum (FBS), and 70% of complete medium, and were stored in liquid nitrogen.

Transient Transfection of Mammalian Cell Lines

bEND5, Py4.1, and NIH 3T3 cells were transfected using Lipofectamine (Invitrogen). 0.5×10⁵ bEND5 or Py4.1 cells, and 1×10⁵ NIH 3T3 cells per well of a 24-well plate were seeded on coverslips overnight to reach the desired density on the day of transfection. Transfection was carried out in Opti-MEM I Reduced Serum Medium (Invitrogen) with a 1:2 ratio of DNA:Lipofectamine, using 0.8 μg DNA and 2 μl Lipofectamine per well. Cells and reagents were increased proportionately for larger culture vessels. DNA:Lipofectamine complexes were incubated with the cells for 4 hours, after which they were replaced with normal medium containing serum. Cells were fixed 24 hours post transfection.

Mouse embryonic fibroblasts (MEFs), isolated from 14.5 day embryos were transfected using either Superfect (Qiagen) or Fugene (Roche). For transfections with Superfect, cells were seeded on coverslips overnight at a density of 1×10⁵ cells/well of a 24-well plate, and were transfected in DMEM without antibiotics and FBS. 1 μg DNA and 5 μl Superfect were incubated with the cells for 3 hours, after which complexes were replaced with normal medium. For transfections with Fugene, the same density of cells was seeded overnight, and transfected in DMEM without antibiotics. 0.2 μg DNA and 0.6 μl Fugene were incubated with the cells, and without replacing the medium, cells were fixed 24 hours post transfection. MEF 3T3 Tet-Off cells were transfected using Superfect (Qiagen) as described above.

In all transfection experiments, an expression plasmid encoding Green Fluorescent Protein (GFP) was used as a control for transfection efficiency.

### Example 2

Fenestrae Induction in Endothelial Cells

Coverslips and dishes were coated with 1% gelatin (Sigma) solution in PBS for 30 minutes at room temperature. Endothelial cells were seeded overnight at a density equivalent to 1.5×10⁶ cells per 100 mm dish. Cultures were induced with Cytochalasin B (Sigma) at 10 μM for 2 hours, with Latrunculin A (Molecular Probes) at 2.5 μM for 3 hours, or with a combination of recombinant mouse 75 ng/ml VEGF (R&D systems) for 6-72 hours and 10 μM Cytochalasin B for 2 hours. Cells were processed for biochemistry or morphology immediately after the end of the induction.

To inhibit protein synthesis during fenestrae formation, cells were incubated with 10 μg/ml Cycloheximide (Sigma) for 30 minutes, and then induced with VEGF (75 ng/ml) for 6 hours and Cytochalasin B (10 μM) for the last 2 hours.

### Example 3

Light Microscopy

Images were captured using the following instruments and software packages:

1) LSM510 laser scanning confocal microscope (Zeiss); 63×1.40 NA Plan-Achromat oil immersion objective

2) TCS SP2 spectral confocal microscope (Leica); 40×1.25 NA Plan-Achromat oil immersion objective; 63×1.4 NA Plan-Achromat oil immersion objective; 100×; Leica confocal software version 2.5

3) Widefield DMRA4 microscope (Leica); orca ER2 camera (Hamamatsu); Metamorph Software (Universal Imaging Corporation)

4) MZFL III Fluorescence Stereomicroscope (Leica); Retiga Camera (Q-Imaging); OpenLab 3.1.7 (Improvision, Inc.)

Digital images were processed using Adobe Photoshop 7.0 (Adobe Systems Inc.)

Incorporation by Reference

The patent and scientific literature referred to herein establishes knowledge that is available to those of skill in the art. All issued patents, patent applications, published foreign applications, and published references, which are cited herein, are hereby incorporated by reference to the same extent as if each was specifically and individually indicated to be incorporated by reference in their entirety.

Equivalents

Those skilled in the art will recognize, or be able to ascertain, using no more than routine experimentation, numerous equivalents to the specific embodiments described specifically herein. Such equivalents are intended to be encompassed in the scope of the following claims.

We claim:

1. A method of inducing the formation of fenestrae in an endothelial cell line selected from the group consisting of a bEND5 endothelial cell line and a Py4.1 endothelial cell line comprising the step of administering an inducing agent selected from the group consisting of latrunculin A and cytochalasin B.

2. The method of claim 1, wherein the endothelial cell line is a bEND5 endothelial cell line.

3. The method of claim 1, wherein the endothelial cell line is a Py4.1 endothelial cell line.

4. The method of claim 1, wherein the inducing agent is latrunculin A.

5. The method of claim 4, wherein latrunculin A is administered at a concentration of about 2.5 nM.

6. The method of claim 1, wherein the inducing agent is cytochalasin B.

7. The method of claim 6, wherein cytochalasin B is administered at a concentration of about 10 nM.

8. The method of claim 1, wherein the inducing agent induces the formation of fenestrae to greater than or equal to 100 fenestrae per cell.

9. The method of claim 1, wherein the inducing agent induces the formation of fenestrae to greater than or equal to 1000 fenestrae per cell.

10. The method of claim 1, wherein the inducing agent induces the formation of fenestrae greater than or equal to 10-fold from an un-induced endothelial cell line.

11. The method of claim 1, wherein the inducing agent induces the formation of fenestrae greater than or equal to 20-fold from an un-induced endothelial cell line.

12. The method of claim 1, wherein the inducing agent induces the formation of fenestrae greater than or equal to 50-fold from an un-induced endothelial cell line.

13. The method of claim 1, wherein the inducing agent induces the formation of fenestrae greater than or equal to 100-fold from an un-induced endothelial cell line.

14. The method of claim 1, wherein the inducing agent induces the formation of fenestrae greater than or equal to 200-fold from an un-induced endothelial cell line.

15. The method of claim 1, wherein the inducing agent induces the formation of fenestrae to greater than or equal to 3 fenestrae per μm².

16. The method of claim 1, wherein the inducing agent induces the formation of fenestrae to greater than or equal to 5 fenestrae per μm².

17. The method of claim 1, wherein the inducing agent induces the formation of fenestrae to greater than or equal to 60 fenestrae per μm².

18. The method of claim 1, wherein the inducing agent induces the formation of fenestrae to about 3.5 fenestrae per μm².

19. The method of claim 1, wherein the inducing agent induces the formation of fenestrae to about 4.5 fenestrae per μm².

20. The method of claim 1, wherein the inducing agent induces the formation of fenestrae to about 5.3 fenestrae per μm².

21. A method of inducing the formation of fenestrae comprising the steps of administering latrunculin A to a bEND5 cell line.

22. A method of inducing the formation of fenestrae comprising the step of administering latrunculin A to a Py4.1 cell line.

23. A method of inducing the formation of fenestrae comprising the steps of administering cytochalasin B to a bEND5 cell line.

24. A method of inducing the formation of fenestrae comprising the step of administering cytochalasin B to a Py4.1 cell line.

25. A method of inducing the formation of fenestrae in a bEND5 cell line comprising the steps of:
a) administering cytochalasin B to the bEND5 cell line; and
b) administering VEGF-A to the bEND5 cell line.

26. A method of inducing the formation of fenestrae in a bEND5 cell line comprising the steps of:
a) administering cytochalasin B to the bEND5 cell line; and
b) administering PMA to the bEND5 cell line.
